(19) United States
(12) Patent Application Publication
(10) Pub. No.: US 2026/0229346 A1
(43) Pub. Date: Aug. 6, 2026

(54) Healthcare AI SaaS platform optimizing patient management and drug safety

(71) Applicant: Brandy Giovanni Robinson, Escondido, CA (US)

(72) Inventor: Brandy Giovanni Robinson, Escondido, CA (US)

(21) Appl. No.: 19/044,639

(22) Filed: Feb. 4, 2025

(51) Int. Cl. G16H 40/20; G16H 10/60; G16H 70/40; G16H 80/00

(52) U.S. Cl. CPC G16H 40/20; G16H 10/60; G16H 70/40; G16H 80/00

(57) ABSTRACT

The invention provides a healthcare SaaS platform that integrates artificial intelligence (AI) tools with traditional patient case management systems to enhance patient care and safety. The platform enables healthcare professionals and patients to interact through functionalities such as adverse event reporting, appointment scheduling, secure messaging, video conferencing, and AI-driven medical data analysis. AI algorithms analyze medical research, safety reports, and clinical trial data, improving diagnostic accuracy and drug safety. The system also supports collaboration among healthcare teams and integrates with external APIs to streamline workflows. By delivering actionable insights and improving decision-making, the platform addresses challenges in patient safety, diagnostic delays, and fragmented information systems, promoting personalized care and better health outcomes.

### FIELD OF THE INVENTION

The present invention generally relates to healthcare technology and more specifically to a SaaS platform integrating patient case management with AI-driven tools for reporting, collaboration, and communication. It enhances patient care and safety through features like adverse event reporting, appointment scheduling, secure messaging, video conferencing, AI-analyzed medical data, and external API integration.

### BACKGROUND OF THE INVENTION

The healthcare industry faces ongoing challenges in ensuring drug safety, improving diagnostic accuracy, and providing personalized treatment plans. Traditional systems for analyzing medical research, patient data, and prescription histories often fail to detect important patterns and trends. This can result in misdiagnoses, treatment delays, and increased risks to patient safety. Additionally, the use of fragmented and outdated tools limits collaboration among healthcare professionals, further worsening these issues.

While advancements in artificial intelligence (AI) have shown promise in addressing these challenges, current AI solutions often lack seamless integration with traditional patient management systems, reducing their effectiveness in practical clinical Settings.

Furthermore, the absence of user-friendly interfaces and comprehensive data analysis tools has hindered the widespread adoption of these technologies in healthcare.

Thus, there is a need for an improved system and method that combines advanced AI capabilities with traditional healthcare workflows to deliver actionable insights, enhance collaboration, and improve patient outcomes.

The present invention is intended to solve the problems associated with conventional devices and methods and provide improvements on these devices.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present invention provides a software platform delivered as a service that integrates traditional patient case management systems with advanced tools for reporting and collaboration driven by artificial intelligence. The platform enables healthcare professionals, organizations, and patients to interact through various functionalities, including adverse event reporting, appointment scheduling, secure messaging, video conferencing, and discussion forums.

The present invention provides access to medical research, safety reports, and clinical trial data analyzed using artificial intelligence, thereby improving patient care and reducing safety risks. Additionally, the platform supports interaction with external application programming interfaces and incorporates features such as artificial intelligence chatbots, clinician notes, and image uploads, offering a seamless and user-friendly experience.

### BRIEF DESCRIPTION OF THE DRAWINGS

FIG. 1 is an illustration of an online platform consistent with various embodiments of the present disclosure, showing the integration of components designed to enhance workflows and facilitate communication and data analysis.

FIG. 2 is a block diagram of a system for facilitating the streamlining of processes, such as healthcare workflows, through the integration of artificial intelligence-driven tools, communication systems, and data management, in accordance with some embodiments.

FIG. 3 is a block diagram of a computing device for implementing the methods disclosed herein, in accordance with some embodiments. The computing device may include components such as a processing unit, system memory, storage devices, input and output devices, and communication connections, enabling the execution of program modules and integration with other systems.

### DETAIL DESCRIPTIONS OF THE INVENTION

All illustrations of the drawings are for the purpose of describing selected versions of the present invention and are not intended to limit the scope of the present Invention.

As a preliminary matter, it will be readily understood by one of ordinary skill in the relevant art that the present disclosure has broad utility and application. As should be understood, any embodiment may incorporate one or more of the aspects and features disclosed herein and may further incorporate one or more of the disclosed functionalities. Furthermore, any embodiment identified as being “preferred” is considered to be part of the best mode contemplated for carrying out the embodiments of the present disclosure. Other embodiments may also be discussed for illustrative purposes to provide a full and enabling disclosure. Moreover, many embodiments, including adaptations, variations, modifications, and equivalent arrangements, will be implicitly disclosed by the embodiments described herein and fall within the scope of the present disclosure.

Accordingly, while embodiments are described herein in detail with reference to one or more specific implementations, it is to be understood that this disclosure is illustrative and exemplary of the present invention and is provided solely to enable a full and enabling disclosure. The detailed descriptions of one or more embodiments are not intended, nor should they be construed, to limit the scope of patent protection afforded in any claims of a patent issuing herefrom. The scope of such patent protection is defined solely by the claims and their equivalents. It is not intended that the scope of patent protection be limited by reading into any claim limitations described herein that do not explicitly appear in the claims.

For example, any sequences or temporal orders of steps in the methods or processes described herein are illustrative and not restrictive. It should be understood that, although steps of various processes or methods may be described or depicted in a specific sequence or temporal order, the steps are not limited to being performed in that order unless explicitly stated otherwise. The steps of such processes or methods may be carried out in various sequences and orders while still falling within the scope of the present disclosure. Accordingly, it is intended that the scope of patent protection be defined by the issued claims rather than the description set forth herein.

Additionally, it is important to note that each term used herein refers to what an ordinary artisan would understand the term to mean based on its contextual use in the present disclosure. To the extent that the meaning of a term as understood by the ordinary artisan differs from any particular dictionary definition, the meaning understood by the ordinary artisan is intended to prevail.

Furthermore, as used herein, “a” and “an” generally denote “at least one” unless the context explicitly dictates otherwise. When used herein to join a list of items, “or” denotes “at least one of the items” but does not exclude a plurality of items, and “and” denotes “all of the items” in the list.

The following detailed description refers to the accompanying drawings, where applicable. Wherever possible, the same reference numbers are used in the drawings and the detailed description to refer to the same or similar elements. While many embodiments of the disclosure are described, modifications, adaptations, and other implementations are possible. For instance, substitutions, additions, or modifications may be made to the elements illustrated in the drawings, and the methods described herein may be modified by substituting, reordering, or adding steps to the disclosed methods. Accordingly, the following detailed description does not limit the disclosure. Instead, the proper scope of the disclosure is defined by the claims and their equivalents. The present disclosure includes headers, which are used for organizational purposes only and should not be construed as limiting the subject matter disclosed under each header.

The present disclosure includes many aspects and features. While many aspects and features are described in the context of methods, systems, and devices for integrating traditional patient case management systems with advanced tools driven by artificial intelligence to improve healthcare workflows, the embodiments of the present disclosure are not limited to this context.

In general, the methods disclosed herein may be performed by one or more computing devices. For example, in some embodiments, the methods may be performed by a server computer in communication with one or more client devices over a communication network, such as the Internet. In other embodiments, the methods may be performed by one or more computing devices, including at least one server computer, at least one client device, at least one network device, at least one sensor, or at least one actuator. Examples of client devices and server computers include, but are not limited to, desktop computers, laptop computers, tablet computers, personal digital assistants, portable electronic devices, wearable computers, smartphones, Internet of Things (IoT) devices, smart appliances, video game consoles, rack servers, supercomputers, mainframe computers, mini-computers, micro-computers, storage servers, application servers (e.g., mail servers, web servers, real-time communication servers, file transfer protocol servers, virtual servers, proxy servers, domain name system servers, etc.), quantum computers, and other similar devices.

Further, the client devices and server computers may execute software applications, including operating systems (e.g., Windows, macOS, Unix, Linux, Android, etc.), to provide a user interface such as a graphical user interface, touch-screen interface, voice-based interface, or gesture-based interface for interaction with users. These devices may also include network interfaces for communication with other devices over a communication network. For example, server computers may include processing devices configured to perform various data processing tasks, such as analyzing, identifying, determining, generating, transforming, calculating, computing, compressing, decompressing, encrypting, decrypting, scrambling, splitting, merging, interpolating, extrapolating, redacting, anonymizing, encoding, and decoding data. Additionally, server computers may include communication devices configured to transmit and receive information in electronic form over wired or wireless communication channels.

The server computers may also include storage devices configured to perform data storage and retrieval operations. These storage devices may incorporate technologies such as data compression, data backup, data redundancy, deduplication, error correction, data fingerprinting, and role-based access control to ensure reliable storage of digital Information.

One or more steps of the methods disclosed herein may be initiated, maintained, controlled, or terminated based on control inputs received from devices operated by users, including end users, administrators, or other stakeholders. The term “user,” as used herein, may refer to a human, an animal, or an artificially intelligent being in any state of existence, unless explicitly stated otherwise in the present disclosure.

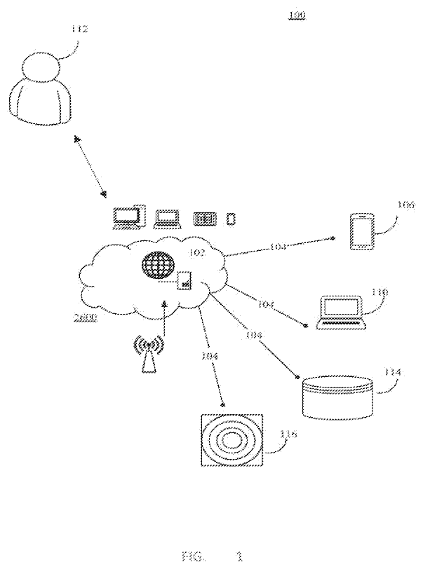

FIG. 1 is an illustration of an online platform 100 consistent with various embodiments of the present disclosure. By way of non-limiting example, the online platform 100 is configured to facilitate the integration of traditional patient case management systems with advanced tools for reporting, collaboration, and communication in healthcare workflows.

The online platform 100 may be hosted on a centralized server 102, such as, for example, a cloud computing service. The centralized server 102 may communicate with other network entities, such as, for example, a mobile device 106 (e.g., a smartphone, a laptop, a tablet computer, etc.), other electronic devices 110 (e.g., desktop computers, server computers, etc.), databases 114, and sensors 116 over a communication network 104, such as, but not limited to, the Internet. Further, the databases 114 may be associated with healthcare organizations, research institutions, or regulatory agencies. Users of the online platform 100 may include relevant parties such as, but not limited to, healthcare professionals, administrators, patients, service providers, and other stakeholders. Accordingly, in some instances, electronic devices operated by one or more relevant parties may be in communication with the platform.

A user 112, such as one or more relevant parties, may access the online platform 100 through a web-based software application or browser. The web-based software application may be embodied as, for example, but not limited to, a website, a web application, a desktop application, or a mobile application compatible with a computing device 2600.

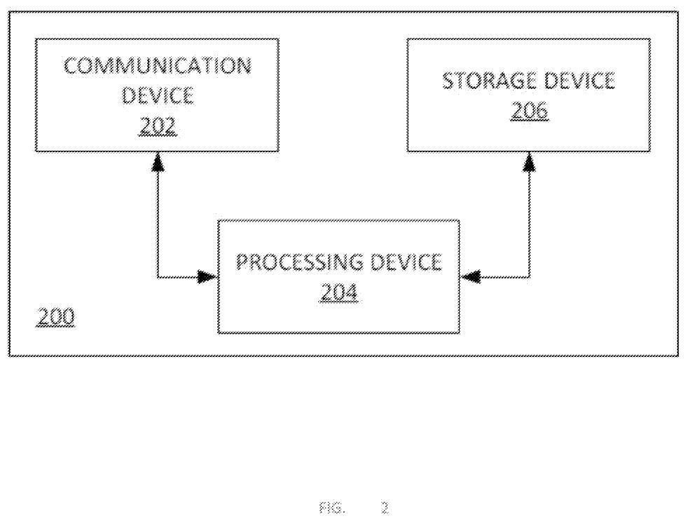

FIG. 2 is a block diagram of a system 200 for facilitating the integration of artificial intelligence-driven tools with traditional patient management systems to improve healthcare workflows, in accordance with some embodiments. Accordingly, the system 200 may include a communication device 202, a processing device 204, and a storage device 206.

Further, the communication device 202 may be configured for transmitting and receiving data between the centralized server and external devices, such as user devices, databases, and sensors. The at least one user device may include, but is not limited to, a smartphone, a tablet, a desktop computer, or a laptop computer. The communication device 202 may also facilitate secure messaging, video conferencing, and interaction with external application programming interfaces to enable seamless collaboration among users.

The present invention is a software platform delivered as a service that utilizes artificial intelligence to enhance clinical practice by analyzing medical research, drug safety information, patient data, and prescription history. The platform integrates traditional patient management systems with tools driven by artificial intelligence, enabling healthcare professionals to identify drug safety concerns, improve diagnostic accuracy, and create personalized treatment plans. With features such as adverse event reporting, video conferencing, secure messaging, and access to research analyzed by artificial intelligence, the platform streamlines workflows and reduces risks to patient safety. It also supports interaction with external application programming interfaces and provides secure portals for doctors, healthcare organizations, and patients, offering a seamless and user-friendly experience.

The present invention comprises a web platform, a mobile application, a secure portal, and a suite of integrated tools and features that collectively enhance healthcare workflows through artificial intelligence-driven analysis, communication, and collaboration. Each component of the invention plays a specific role and is designed to integrate seamlessly with other components to create a unified, user-friendly experience for healthcare professionals, enterprises, and patients.

### Web Platform

This component of the present invention is a secure, browser-based interface designed for healthcare professionals and patients to access the platform's features. The web platform serves as the central hub, connecting with the secure portal, mobile application, and backend database. It also integrates with external systems through application programming interfaces, enabling data exchange and real-time updates.

### Mobile Application

This component of the present invention is a companion application providing on-the-go access to the platform's features for healthcare professionals and patients. The mobile application synchronizes with the web platform and secure portal, allowing users to access real-time updates, schedule appointments, and communicate seamlessly across devices.

### Secure Portal

The present invention also includes separate portals tailored for doctors, enterprises, and patients, offering features specific to their roles.

Enterprise Portal may provide tools for patient and doctor management, AI-analyzed research, clinical trial information, adverse reporting, and safety reports.

Doctor Portal may provide tools for patient management, AI-analyzed research, adverse reporting, safety reports, clinical notes, clinical trial information, video conferencing, and messaging.

Patient Portal may offer appointment management, messaging, video conferencing, adverse reporting, clinical trial information, access to AI-analyzed safety reports and read-only access to community forums.

This secure portal can be linked to the web platform and mobile application, ensuring consistent user experiences across devices. It may also connect to external databases and APIs for retrieving medical research and safety reports.

### Tools and Features

AI-Driven Analysis: In one embodiment, the present invention may use advanced algorithms to analyze medical research, drug safety information, patient data, and prescription history. The AI tools may be embedded in the web platform, mobile application, and secure portal, providing actionable insights across all components.
Video Conferencing and Messaging Systems: In one embodiment, the present invention may facilitate real-time communication between users through video conferencing and messaging systems. These systems may be accessible through both the web platform and mobile application, ensuring seamless communication regardless of the device used.
Internal AI Chatbot: In another embodiment, the present invention may provide an automated AI chatbot for assistance and query resolution. The chatbot may interact with the AI analysis tools, secure portal, and communication systems to offer context-specific support.
Clinical Notes and Research Tools: In yet another embodiment, the present invention may enable healthcare professionals to document patient information and access medical research. These tools may be linked to the AI analysis system, ensuring that entered data contributes to improved insights and recommendations.
Adverse Event and Safety Reporting Tools: In yet another embodiment, the present invention may allow users to identify and report drug safety concerns through adverse event and safety reporting tools. These tools may be connected to the AI system and external APIs, enabling real-time reporting and updates.
Patient History and Data Management: In yet another embodiment, the present invention may store and analyze patient records for improved decision-making. This component may be integrated with the secure portal and AI tools, ensuring that all data is accessible and actionable.
Image Capture and Upload: In yet another embodiment, the present invention may allow users to add visual data to patient profiles through image capture and upload functionality. This feature may be linked to the patient history system and clinical notes, ensuring that uploaded images are securely stored and analyzed.
Calendar Scheduling and Appointment Management: In yet another embodiment, the present invention may organize and manage appointments for users through calendar scheduling and appointment management tools. This tool may be connected to the secure portal and mobile application, providing synchronized scheduling across devices.

The components of the present invention are designed to integrate seamlessly, creating an efficient and user-friendly platform. In one embodiment, the integration can be achieved as follows:

In preferred embodiment, all data entered through the web platform, mobile application, or secure portal is stored in a centralized database. This ensures that users can access the same information regardless of the device or interface used.

The AI analysis tools continuously process data from patient records, prescription histories, and clinical notes. These insights are made available to users through the secure portal, web platform, and mobile application, ensuring real-time access to actionable information.

In some embodiments, video conferencing, messaging, and discussion forums are accessible across doctor-patient communities and components, enabling seamless communication between healthcare professionals and patients and privacy of information between healthcare professionals and patients. These tools also integrate with external APIs to facilitate interaction with third-party systems.

The secure portal may provide tailored access for doctors, enterprises, and patients. Each portal is linked to the AI tools, communication systems, and scheduling features, ensuring that users can perform their tasks efficiently.

In some embodiments, the platform may connect to external databases, APIs, and third-party systems to retrieve medical research, safety reports, and clinical trial data. This integration enhances the platform's capabilities and ensures that users have access to comprehensive information.

The present invention is assembled with a user-friendly interface that may include the following:

Login Page: A healthcare-themed entry point guiding users to their respective portals.

Doctor Portal: A dashboard providing tools for patient management, AI-analyzed research, adverse reporting, safety reports, clinical notes, clinical trial information, video conferencing, messaging, and appointment scheduling.

Enterprise Portal: A dashboard providing tools for patient management, including AI-analyzed research, safety reports, clinical trial information, and adverse Reporting.

Patient Portal: A simplified dashboard offering appointment management, messaging, video conferencing, adverse reporting, clinical trial information, and AI-analyzed safety reports.

Each portal is designed to display summarized activity, trends, and insights tailored to the user type.

In some embodiments, the platform may streamline healthcare workflows as follows:
A healthcare professional logs into the enterprise portal.
The professional accesses the enterprise dashboard to:
   Add and manage doctor and patient information.
   View clinical trial information, adverse reporting, AI-analyzed safety reports and medical research.
A healthcare practitioner logs into the doctor portal.
The practitioner accesses the doctor dashboard to:
   Add and manage patient information.
   View AI-analyzed safety reports, clinical trial information, and medical research.
   Document clinical notes and report adverse events.
   Communicate with patients or colleagues through video conferencing and messaging.
   Communicate with colleagues through community discussion forums.
Patients can log into their patient portal to:
   Request and view appointment details and patient information.
   Update patient information
   Communicate with their doctor through messaging or video conferencing.
   Access clinical trial information, adverse reporting, AI-analyzed safety reports and community forums.
The AI system analyzes the entered data, providing actionable insights to improve diagnostic accuracy and personalize treatment plans.
As more data is added, the AI system continuously fine-tunes its analysis, ensuring increasingly accurate results.

In one embodiment the present invention may provide a method for streamlining healthcare workflows and improving patient outcomes through an integrated platform, the method may include various steps comprising: enabling a healthcare professional or patient to access the system via a web platform or mobile application; logging the user into a secure portal tailored for healthcare professionals, enterprises, or patients; allowing healthcare professionals to input patient data, prescription history, and clinical notes into the system; enabling patients to manage appointments and communicate with healthcare providers through the patient portal; analyzing entered data, including medical research, drug safety information, and patient records, using artificial intelligence algorithms to identify patterns and trends; continuously refining the artificial intelligence analysis as additional data is entered into the system; enabling users to communicate through video conferencing, messaging, or discussion forums; allowing healthcare professionals to collaborate with colleagues or patients using integrated communication tools; enabling healthcare professionals to identify and report drug safety concerns using adverse event reporting tools; generating and presenting artificial intelligence-analyzed safety reports for review; securely storing all data in a centralized database accessible across all components of the system; allowing users to retrieve stored data for decision-making or patient care; interacting with external databases and application programming interfaces to retrieve additional information, including clinical trial data and updated medical research; refining artificial intelligence algorithms based on newly added data to improve the accuracy of insights and recommendations over time; providing healthcare professionals with actionable insights to enable more accurate diagnoses and the development of personalized treatment plans; and enhancing patient outcomes by improving communication, providing access to safety reports, and streamlining appointment management.

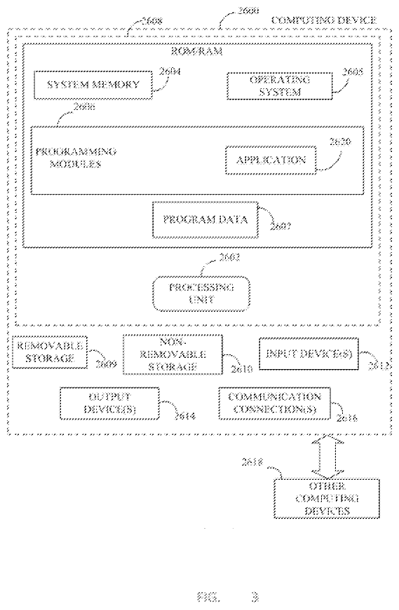

With reference to FIG. 3, a system consistent with an embodiment of the present disclosure may include a computing device or cloud-based service, such as computing device 2600. In its basic configuration, computing device 2600 may include at least one processing unit 2602 and a system memory 2604. Depending on the configuration and type of computing device, system memory 2604 may comprise, but is not limited to, volatile memory such as random-access memory, non-volatile memory such as read-only memory, flash memory, or any combination thereof. System memory 2604 may include an operating system 2605, one or more programming modules 2606, and program data 2607. For example, operating system 2605 may control the operation of computing device 2600, while programming modules 2606 may include components such as an image-processing module, a machine learning module, or other modules for performing specific tasks. Program data 2607 may include data used or generated by these modules. This basic configuration is illustrated in FIG. 3 by the components within dashed line 2608.

Computing device 2600 may also include additional features or functionality. For instance, it may include additional data storage devices, both removable and non-removable, such as magnetic disks, optical disks, or tape. These additional storage devices are illustrated in FIG. 3 as removable storage 2609 and non-removable storage 2610. Examples of computer storage media include, but are not limited to, volatile and non-volatile memory, removable and non-removable media, and any technology capable of storing information such as computer-readable instructions, data structures, program modules, or other data. Examples of such media include random-access memory, read-only memory, electrically erasable programmable read-only memory, flash memory, compact discs, digital versatile discs, magnetic cassettes, magnetic tape, magnetic disk storage, or any other medium that can store information and be accessed by computing device 2600. Any such computer storage media may form part of computing device 2600.

Computing device 2600 may also include input devices 2612, such as a keyboard, mouse, pen, sound input device, touch input device, location sensor, camera, biometric sensor, or other input mechanisms. Output devices 2614, such as a display, speakers, printer, or other output mechanisms, may also be included. These input and output devices are examples, and other devices may be used.

Additionally, computing device 2600 may include a communication connection 2616 that allows it to communicate with other computing devices 2618 over a network, such as an intranet or the Internet, in a distributed computing environment. Communication connection 2616 is an example of communication media, which may include computer-readable instructions, data structures, program modules, or other data in a modulated data signal, such as a carrier wave or other transport mechanism. Communication media may include wired media, such as a wired network or direct-wired connection, and wireless media, such as acoustic, radio frequency, infrared, or other wireless media. The term “computer-readable media” as used herein includes both storage media and communication media.

As described above, system memory 2604 may store a number of program modules and data files, including operating system 2605. While executing on processing unit 2602, programming modules 2606 may perform processes such as one or more stages of methods, algorithms, systems, applications, servers, or databases as described in the present disclosure. For example, programming modules 2606 may include machine learning applications or other modules for analyzing data, generating insights, or facilitating communication. These processes are examples, and processing unit 2602 may perform other processes as well.

Program modules, as used in the present disclosure, may include routines, programs, components, data structures, and other types of structures that perform specific tasks or implement particular abstract data types. Embodiments of the disclosure may be practiced with various computer system configurations, including handheld devices, general-purpose graphics processor-based systems, multiprocessor systems, microprocessor-based or programmable consumer electronics, application-specific integrated circuit-based electronics, minicomputers, mainframe computers, and others. The disclosure may also be practiced in distributed computing environments where tasks are performed by remote processing devices linked through a communications network. In such environments, program modules may be located in both local and remote memory storage devices.

Furthermore, embodiments of the disclosure may be implemented in an electrical circuit comprising discrete electronic elements, packaged or integrated electronic chips containing logic gates, a circuit utilizing a microprocessor, or a single chip containing electronic elements or microprocessors. The disclosure may also be practiced using other technologies capable of performing logical operations, such as mechanical, optical, fluidic, or quantum technologies. Additionally, embodiments may be implemented within a general-purpose computer or other circuits or systems.

Embodiments of the disclosure may be implemented as a computer process (method), a computing system, or an article of manufacture, such as a computer program product or computer-readable media. The computer program product may include computer storage media readable by a computer system and encoding a computer program of instructions for executing a computer process. Alternatively, the computer program product may include a propagated signal on a carrier readable by a computing system and encoding a computer program of instructions for executing a computer process. Accordingly, the present disclosure may be embodied in hardware, software (including firmware, resident software, or microcode), or a combination thereof.

In other words, embodiments of the present disclosure may take the form of a computer program product on a computer-usable or computer-readable storage medium containing program code for use by or in connection with an instruction execution system. A computer-usable or computer-readable medium may include any medium capable of containing, storing, communicating, propagating, or transporting the program for use by or in connection with the instruction execution system, apparatus, or device. Examples of such media include, but are not limited to, an electrical connection with one or more wires, a portable computer diskette, random-access memory, read-only memory, erasable programmable read-only memory, flash memory, optical fiber, portable compact disc read-only memory, or other suitable media.

While certain embodiments of the disclosure have been described, other embodiments may exist. Additionally, although embodiments of the present disclosure have been described as being associated with data stored in memory and other storage media, data may also be stored on or read from other types of computer-readable media, such as secondary storage devices like hard disks, solid-state storage, compact discs, or carrier waves from the Internet. Furthermore, the stages of the disclosed methods may be modified in any manner, including reordering stages or inserting or deleting stages, without departing from the scope of the disclosure.

Although the invention has been explained in relation to its preferred embodiment, it is to be understood that many other possible modifications and variations can be made without departing from the spirit and scope of the invention.

1. A software platform for enhancing healthcare workflows through the integration of artificial intelligence-driven tools with traditional patient management systems, comprising:
A centralized server configured to communicate with user devices, external databases, and sensors over a communication network;
A web platform providing a secure, browser-based interface for healthcare professionals, administrators, patients, and other stakeholders to access features of the platform;
A mobile application that synchronizes with the web platform, allowing healthcare professionals and patients to access features of the platform on mobile devices;
A secure portal tailored for different user roles, including healthcare professionals, enterprises, and patients, offering features such as patient management, adverse event reporting, messaging, video conferencing, and access to AI-analyzed medical research;
An artificial intelligence system configured to analyze medical research, drug safety data, patient data, and prescription history to generate actionable insights and improve decision-making within healthcare workflows;
Integration with external databases, application programming interfaces (APIs), and third-party systems to retrieve medical research, safety reports, and clinical trial data, enhancing the platform's capabilities and ensuring comprehensive information access.

2. The software platform of claim 1, wherein the secure portal comprises separate access interfaces for:
Healthcare professionals, providing tools for patient management, clinical notes, adverse event reporting, video conferencing, and AI-analyzed research;
Enterprises, providing tools for managing patients, doctors, clinical trial data, adverse reporting, and safety reports;
Patients, providing tools for appointment management, messaging, adverse event reporting, clinical trial information, and access to AI-analyzed safety reports.

3. The software platform of claim 1, wherein the AI-driven tools provide clinical decision support by integrating with patient history data and offering insights based on analysis of medical records, research, and safety information.

4. The software platform of claim 1, wherein the platform is configured to display summarized activity, trends, and insights tailored to each user type, including healthcare professionals, administrators, and patients.

5. The software platform of claim 1, further comprising:
A Doctor Portal providing a dashboard that includes patient management tools, AI-analyzed research, adverse reporting, safety reports, clinical notes, clinical trial information, video conferencing, messaging, and appointment scheduling;
An Enterprise Portal providing a dashboard for managing doctor and patient information, clinical trial data, adverse reporting, AI-analyzed research, and safety reports;
A Patient Portal providing a simplified dashboard for appointment management, messaging, video conferencing, adverse reporting, clinical trial information, and AI-analyzed safety reports.

6. The software platform of claim 1, wherein the AI system is embedded within the web platform, mobile application, and secure portal, providing real-time analysis of medical data to assist healthcare professionals in identifying drug safety concerns, improving diagnostic accuracy, and generating personalized treatment plans.

7. The software platform of claim 1, wherein the platform is configured to integrate with external databases and APIs to retrieve medical research, safety reports, and clinical trial data from third-party systems, ensuring comprehensive and up-to-date information access for users.

8. The software platform of claim 1, wherein the system comprises a set of workflows to streamline healthcare processes, including:
A healthcare professional logging into the Enterprise Portal to manage doctor and patient information, view clinical trial data, adverse reporting, and AI-analyzed safety reports;
A healthcare practitioner logging into the Doctor Portal to manage patient information, document clinical notes, report adverse events, and communicate with patients and colleagues via video conferencing or messaging;
A patient logging into the Patient Portal to manage appointments, communicate with their doctor, access clinical trial information, and view AI-analyzed safety reports.

9. The software platform of claim 1, wherein the AI system analyzes entered data, including medical research, drug safety information, and patient records, to identify patterns and trends, continuously refining the analysis as additional data is entered into the system.

10. The software platform of claim 1, wherein the platform provides tools for adverse event and safety reporting, allowing users to report drug safety concerns in real-time, with integration to external databases and APIs for live updates on safety reports and clinical trials.

11. The software platform of claim 1, further comprising a communication device configured to facilitate secure messaging and video conferencing between users of the platform and to interact with external APIs for seamless collaboration.

12. The software platform of claim 1, wherein the platform includes a calendar scheduling and appointment management tool, allowing users to organize and manage appointments across devices, synchronized with the secure portal and mobile application.

13. A method for streamlining healthcare workflows and improving patient outcomes through an integrated platform, comprising:
Enabling a healthcare professional or patient to access the system via a web platform or mobile application;
Logging the user into a secure portal tailored for healthcare professionals, enterprises, or patients;
Allowing healthcare professionals to input patient data, prescription history, and clinical notes into the system;
Enabling patients to manage appointments and communicate with healthcare providers through the patient portal;
Analyzing entered data, including medical research, drug safety information, and patient records, using artificial intelligence algorithms to identify patterns and trends;
Continuously refining the artificial intelligence analysis as additional data is entered into the system;
Enabling users to communicate through video conferencing, messaging, or discussion forums;
Allowing healthcare professionals to collaborate with colleagues or patients using integrated communication tools;
Enabling healthcare professionals to identify and report drug safety concerns using adverse event reporting tools;
Generating and presenting artificial intelligence-analyzed safety reports for review;
Securely storing all data in a centralized database accessible across all components of the system;
Allowing users to retrieve stored data for decision-making or patient care;
Interacting with external databases and application programming interfaces (APIs) to retrieve additional information, including clinical trial data and updated medical research;
Refining artificial intelligence algorithms based on newly added data to improve the accuracy of insights and recommendations over time;
Providing healthcare professionals with actionable insights to enable more accurate diagnoses and the development of personalized treatment plans;
Enhancing patient outcomes by improving communication, providing access to safety reports, and streamlining appointment management.

14. The method of claim 13, wherein the platform retrieves medical research, safety reports, and clinical trial data from external databases, APIs, and third-party systems, enhancing the platform's capabilities and ensuring comprehensive information access.

15. The method of claim 13, further comprising:
Providing a Doctor Portal for healthcare professionals to manage patient information, document clinical notes, access AI-analyzed safety reports, and communicate with patients;
Providing an Enterprise Portal for healthcare administrators to manage doctor and patient data, clinical trial information, and adverse event reports;
Providing a Patient Portal for patients to manage appointments, update patient information, and access clinical trial data and safety reports.

16. The method of claim 13, wherein the AI system continuously fine-tunes its analysis to improve the accuracy of diagnoses and treatment recommendations as more data is added, including insights from external databases and APIs.

17. The method of claim 13, wherein the platform enables real-time communication between healthcare professionals, patients, and colleagues via video conferencing, messaging, and community discussion forums, supporting collaboration and decision-making.

18. The method of claim 13, further comprising providing users with access to a calendar and scheduling tool for managing and organizing appointments, synchronized with the doctor and patient portals.

19. A computing device for executing the platform of claim 1, comprising:
A processing unit;
A system memory including an operating system and program modules for executing methods as described in claim 13;
A storage device configured to store data used or generated by the platform;
A communication connection to enable communication with other computing devices over a network.

20. A computer-readable medium storing instructions for executing the method of claim 13, wherein the instructions are configured to cause a computing device to perform operations that integrate AI-driven tools into healthcare workflows, improve decision-making, and enhance patient outcomes.
